(19) United States
(12) Patent Application Publication
(10) Pub. No.: US 2004/0253290 A1
(43) Pub. Date: Dec. 16, 2004

(54) Method for preparing bioabsorbable organic/inorganic composition for bone fixation devices and itself prepared thereby

(76) Inventors: Kyeong Ah Kim, Kyungki-do (KR); Byung Kun Son, Seoul (KR); Yong Gun Jeon, Kyungki-do (KR)

Correspondence Address: D. PETER HOCHBERG CO. L.P.A., 1940 EAST 6TH STREET, CLEVELAND, OH, 44114, US

(21) Appl. No.: 10/481,905

(22) PCT Filed: Jun. 29, 2002
(86) PCT No.: PCT/KR02/01199

(30) Foreign Application Priority Data
Jun. 29, 2001 (KR) 2001/38338

(51) Int. Cl. A61F 2/00

(52) U.S. Cl. 424/423

(57) ABSTRACT

A high-strength, biodegradable, organic polymer/inorganic particle composite material for bone fixation, which is prepared by mixing and dispersing a biocompatible, inorganic fine or ultrafine particle in an organic monomer and then polymerizing the organic monomer and thus exhibits remarkably improved mechanical strength, and also to a high-strength, biodegradable, organic polymer/inorganic particle composite material prepared thereby is disclosed. More particularly, the a high-strength, biodegradable, organic polymer/inorganic particle composite material for bone fixation is prepared by mixing and dispersing a calcium phosphorus compound or a calcium aluminate compound in a biodegradable organic monomer, at the amount of 0.5 to 60% by weight; and polymerizing the biodegradable organic monomer; and then forming the polymerized material into a desired shape. The synergistic, reinforcing effect of the inorganic fine particle is increased, so that the high-strength material for bone fixation can be prepared. Also, the biocompatible fine particle is used, so that a long-term side effect can be reduced.

### TECHNICAL FIELD

The present invention relates to a manufacturing method of a high-strength, biodegradable, organic polymer/inorganic particle composite material for bone fixation, and also to a high-strength, biodegradable, organic polymer/inorganic particle composite material for bone fixation manufactured thereby. More particularly, the present invention relates to a high-strength, biodegradable, organic polymer/inorganic particle composite material for bone fixation, which is manufactured by mixing and dispersing a biocompatible, inorganic fine or ultrafine particle in an organic monomer and then polymerizing the organic monomer and thus exhibits remarkably improved mechanical strength, and also to a high-strength, biodegradable, organic polymer/inorganic particle composite material manufactured thereby.

### BACKGROUND ART

Implant materials are used for patients who received a wound at the face, the cranium or the bone tissue of various sites of the human body. Such implants are generally formed of three kinds of materials, i.e., a metallic material, a ceramic material and an organic polymer material. Specifically, the metallic material is excellent in mechanical strength, such as tensile strength and compression strength, and can be processed into a desired shape by means of the conventional processing method, including cutting, casting and simple deformation. Also, it is relatively stable against the chemical reaction in the body. However, it has a disadvantage in that it can cause a stress-protection effect at a newly growing tissue, as the load applied to bone tissue by a hard metal support is reduced. Another disadvantage is that, when it is mounted in the body for a long period of time, the breakage caused by partial corrosion can occur.

Meanwhile, the ceramic material has a great affinity to bone. However, it is disadvantageous in that it has low impact strength, and cannot be deformed after it was formed into an implant, so that the ceramic material is difficult to be deformed at the scene of a surgical operation and also coped with the bone recovery during a treatment period are difficult.

Finally, the organic polymer material has advantages, including excellent impact strength, high affinity to tissue and the easiness of processing, and thus various biodegradable materials were developed. Examples of the biodegradable materials include polylactide (PLA), such as poly-L-lactide and poly-L/DL-lactide, polyglycolide (PGA), polycaprolactone (PCL) and polydioxanone. However, these materials have a problem in that the strength and stiffness required to support bone are insufficient.

As methods for solving this insufficient strength problem of the biodegradable polymer materials, a self-reinforcing method and a solid-state extrusion method were known, but the resulting materials have no satisfactory strength. Another method for solving the insufficient strength of the biodegradable polymers was disclosed in U.S. Pat. No. 4,781,183 in which a hydroxyapatite particle as a biodegradable inorganic ceramic material for reinforcing was introduced at the final step of melt polymerization for forming polylactide. In addition, a method was known in which the composite material was prepared by melt-blending, mixing and solid-state mixing of the polymer and the biodegradable inorganic ceramic material. However, in such methods, the biodegradable polymers showed a rapid reduction in molecular weight at a melt or solution state, so that the mechanical strength of a desired level cannot be obtained. Furthermore, the dispersion degree of the inorganic particle, such as ceramic particles, in the polymer, is absolutely critical to obtain the reinforcing effect caused by the mixing of inorganic particle. However, with this simple mixing method, the particle dispersibility of a high level cannot be obtained due to the resistance caused by the viscosity of the polymer.

As a result, the studies reported up to now on the binding of the ceramic material with the organic polymer were mostly to simply mix the ceramic particle with the biodegradable polymer, or to reinforce the biodegradable polymer with ceramic fibers. The results of these studies were significantly different from the level realizing the ultimate end of the bone fixation material.

Meanwhile, U.S. Pat. No. 4,655,777 discloses a method in which the inorganic ceramic fibers were laminated or solution-coated on the organic polymer. However, in this method, it was difficult to avoid the reduction in molecular weight during a preparation process, and the remarkable improvement in strength was not achieved due to defects, such as bubbles, that occured during the preparation process. Moreover, the resulting fiber-reinforced composite material cannot be subjected to an additional procedure for improving strength, such as a drawing procedure.

### DISCLOSURE OF INVENTION

Accordingly, the present invention has been made to solve the above-mentioned problems occurring in the prior art, and an object of the present invention is to provide a method for manufacturing a high-strength, biodegradable material for bone fixation, which exhibits a remarkable improvement in the dispersibility of a biocompatible, fine or ultrafine particle in an organic polymer material.

Another object of the present invention is to provide a method for easily manufacturing an ideal material for bone fixation, in which a bioabsorbable and biocompatible material is used as an inorganic particle for reinforcement, so that the recovery of injured bone tissue can be promoted and a long-term side effect can be reduced.

To achieve the above objects, the present invention provides a method for manufacturing a high-strength, biodegradable, organic polymer/inorganic particle composite material for bone fixation, which comprises the steps of: mixing and dispersing a biocompatible inorganic fine particle having an average particle size of less than 2 μm in a biodegradable organic monomer, polymerizing the biodegradable organic monomer, thereby preparing a biodegradable organic polymer/inorganic particle composite; and forming the biodegradable organic polymer/inorganic particle composite into a desired shape.

The biodegradable organic monomer is preferably one selected from the group of consisting of glycolide, DL-lactide, L-lactide, caprolactone, dioxanone, esteramide, oxalate and mixtures thereof and is more preferably L-lactide. The inorganic fine particle is preferably a calcium phosphorus compound or a calcium aluminate compound. The calcium phosphorus compound is preferably selected from the group consisting of hydroxyapatite (HA), tricalcium phosphate (TCP), and calcium metaphosphate (CMP). The content of the inorganic fine particle is preferably 0.5 to 60% by weight relative to the weight of the monomer.

The above polymerization step is preferably carried out using dispersion polymerization, suspension polymerization, bulk polymerization or melt polymerization. If the dispersion or suspension polymerization is used, an alkyl/aryl-substituted siloxane compound or a long-chain saturated hydrocarbon compound is preferably used as a dispersant.

The forming step is carried out using injection molding, compression molding, solid-state extrusion or a combination thereof. If the biodegradable organic polymer is poly-L-lactide, the compression molding is preferably carried out at a temperature of 150 to 240° C.

The solid-state extrusion is preferably carried out using hydrostatic extrusion, ram extrusion or die drawing. If the biodegradable organic polymer is poly-L-lactide, the solid-state extrusion is preferably conducted at a temperature of 130 to 150° C. and a drawing ratio of 5 to 12 times.

Hereinafter, the present invention will be described in detail.

The present invention relates to the manufacture of a high-performance, biodegradable material for bone fixation, which comprises the steps of: mixing and dispersing a biocompatible inorganic fine particle having an average particle size of less than 2 μm, at the amount of 0.5 to 60% by weight in a biodegradable organic monomer selected from the group consisting of glycolide, DL-lactide, L-lactide, caprolactone, dioxanone, esteramide, oxalate and mixtures thereof; polymerizing the biodegradable organic monomer, thereby producing a polymeric composite of high strength and high polymerization degree, in which the inorganic fine particle is uniformly dispersed, so that the composite shows a remarkable improvement in its mechanical strength; and subjecting the polymeric composite to a series of manufacturing procedures.

The biodegradable organic polymer prepared in the present invention is aliphatic polyesters, including polyglycolide, poly-DL-lactide, poly-L-lactide, polycaprolactone, polydioxanone, polyesteramide, copolyoxalate (copolymer of polyoxalate), and mixtures thereof, and has a weight-average molecular weight of more than 50,000.

The inorganic fine particle serves as a reinforcement material to reinforce the insufficient strength of the biodegradable polymer material. In the present invention, a calcium phosphorus compound or a calcium aluminate compound can be generally used as the bioabsorbable and biocompatible material. Typical examples of calcium phosphorus include hydroxyapatite, tricalcium phosphate and calcium metaphosphate. Hydroxyapatite (HA) is chemically structurally highly similar to bone of the human body so that it has an excellent bioaffinity. The biocompatible inorganic fine particle of the present invention has an average particle size of less than 2 μm more preferably of less than 50 nm. For this reason, it is advantageous in that it promotes the rapid adaptation of bone to an implant and prevents the thick fibrous tissue from occurring around the implant. In addition, it firms the binding of the implant to bone and thus reduces the treatment period.

Moreover, tricalcium phosphate (TCP) and calcium metaphosphate (CMP) are biodegradable ceramic materials, which are sufficiently bound to biotissue at an early stage upon in vivo implantation, and then gradually degraded and lost.

In addition, the calcium aluminate compound also exhibits various crystalline forms and in vivo absorption behaviors depending on the ratio of calcium and aluminum. CaAl₂SO₄ which is a typical example of the calcium aluminate shows an absorption rate of 60% at one year after in vivo implantation, so that it is useful as an implant material.

The present invention relates to a method for manufacturing a high-strength material for bone fixation, in which the reinforcing effect of the inorganic fine particle is increased to the maximum. According to the present invention, a method was developed, which permits increasing the reinforcing effect by improving the dispersibility of the inorganic fine particle in the biodegradable organic polymer. The reinforcing effect of the inorganic fine particle greatly varies depending on the size of the reinforcement material and the dispersibility of the reinforcement material in the polymer. The smaller the size of the reinforcement material (i.e., order of millimeter (mm)<micrometer(μm)<nanometer(nm)), the reinforcement effect is increased. The higher the dispersibility of the particle in the polymer, the reinforcing effect is increased. On the other hand, the smaller the size of the particle, the uniform dispersion of the particle in the polymer is difficult. If the viscosity of the polymer upon mixing is low, the dispersibility of the particle will be improved.

Accordingly, as the reinforcing inorganic particle used in the present invention, a fine particle of a micrometer size, particularly a calcium phosphorus compound (Ca—P) or a calcium aluminate compound (Ca—Al) having an average particle size of less than 2 μm is introduced together with the biodegradable organic monomer, thereafter which the biodegradable organic monomer is polymerized. Thus, the dispersibility of the inorganic particle can be greatly improved.

In this case, the content of the inorganic particle is preferably 0.5 to 60% by weight, and more preferably 5 to 40% by weight, relative to the weight of the biodegradable organic monomer. If the content of the inorganic fine particle is less than 0.5% by weight, the reinforcing effect will be insufficient. If the content of the inorganic particle is more than 60% by weight, the reinforcing effect will be lowered and impact resistance will be rapidly reduced.

The inorganic calcium compound as the inorganic fine particle can be produced by a three-step process consisting of (1) the preparation of a solution, (2) the synthesis of a precursor and (3) heat treatment, as known in the art.

For example, hydroxyapatite can be prepared by ultrasonically dispersing a fine particle produced from a aqueous solution mixture where an aqueous calcium nitrate solution and an aqueous ammonium phosphorus solution were mixed with each other at a desired ratio; and then drying and heat-treating the resulting dispersion.

Also, the tricalcium phosphate particle can be prepared by a known Salsbury and Doremus method which enables tricalcium phosphate of high purity to be obtained. In other words, the tricalcium phosphate can be prepared as follows. Ammonia water is added to an aqueous calcium nitrate solution and diluted with distilled water so as to prepare a basic aqueous solution. An aqueous ammonium phosphorus solution is also prepared in the same manner. The prepared phosphorus solution is added dropwise to the prepared calcium nitrate solution, centrifuged and washed to give a white sludge to which a dilute ammonium sulfate solution is then added and dispersed. The resulting dispersion is spray-dried and heat-treated, thereby producing the tricalcium phosphate particle.

The calcium metaphosphate can be produced as follows. Calcium carbonate (CaCO₃) is dissolved slowly in an aqueous phosphorus solution, and an aqueous solution of 1-pyrrolidone dithiocarbamate is added to precipitate and remove impurities. The resulting solution is spray-dried and heat-treated.

The calcium-aluminate compound is commercially available in various products depending on the atomic ratio of calcium/alumina. These products can be finely powdered and distributed before use.

Before conducting the polymerization reaction for forming the biodegradable organic polymer, the prepared inorganic fine particle is mixed with, and uniformly dispersed in the biodegradable monomer. Thereafter, a catalyst is added to the mixture, and the polymerization of the monomer is then carried out to give an organic polymer/inorganic particle composite where the inorganic fine particle is uniformly dispersed in the polymer.

In the polymerization step, dispersion polymerization, suspension polymerization, bulk polymerization or melt polymerization can be used. In the case of the dispersion or suspension polymerization, an alkyl/aryl-substituted siloxane compound or a long-chain saturated hydrocarbon compound is used as a dispersant, and an organic tin compound as a catalyst is added, before initiating the polymerization reaction.

The prepared organic polymer/inorganic particle composite is washed and dried to remove any unreacted monomer, low-molecular weight polymer and remaining catalyst. The resulting composite is formed into a desired shape, thereby producing the biodegradable, organic polymer/inorganic particle composite material for bone fixation according to the present invention.

The forming step can be conducted using an injection molding method, a compression molding method, a solid-state extrusion method and a combination thereof.

The compression molding is generally carried out at the temperature range of ±30° C. from the melting point of the polymer for about 30 minutes to 3 hours, although the compression molding temperature varies depending on the kind of the biodegradable organic polymer. In the case of poly-L-lactide used in Example of the present invention, the compression molding is preferably carried out at a temperature of 150 to 240° C., and more preferably at a temperature of 170 to 220° C.

The compression-molded composite can be extruded by an extrusion method, including hydrostatic extrusion, ram extrusion and die drawing. In the present invention, a billet is formed and inserted into a die. Once the temperature of oil surrounding the billet reaches a suitable level, and the billet is applied with hydrostatic pressure and extruded in a solid state by applying the pulling force from the outside. In this case, the solid-state extrusion is preferably carried out at a temperature ranging from the glass transition temperature to melting point of the biodegradable organic polymer and a drawing ratio of 2 to 12 times.

In the case of poly-L-lactide, it is particularly preferred that the extrusion temperature is in the range of 130 to 150° C., and the drawing ratio is in the range of 5 to 12 times.

### BEST MODE FOR CARRYING OUT THE INVENTION

The present invention will hereinafter be described in further detail by examples. It should however be borne in mind that the present invention is not limited to or by the examples.

### EXAMPLE 1

120 g of L-lactide as a matrix material, and 6 g of tricalcium phosphate (TCP) of less than 50 nm as an inorganic fine particle, were introduced into a flask, and depressurized under vacuum for 4 hours or above to remove air and impurities within the flask. Then, the mixture was heated to a temperature of 100 to 120° C. to completely melt the L-lactide. The flask was introduced into a heated ultrasonic vessel for washing, and the tricalcium phosphate particle was sufficiently dissolved in the melted L-lactide. Then, 300 ml of silicon oil of a 10 cSt viscosity was introduced into the reaction flask, while maintaining the flask at a vacuum condition. The internal temperature of the flask was heated to 130° C., and 0.27 ml of a 20-fold toluene dilution of stannous octoate as a catalyst was introduced into the flask by means of an injector, after which polymerization was carried out for 24 hours. The obtained poly-L-lactide/tricalcium phosphate composite powder was filtered, washed several times with hexane to remove remaining silicon oil, washed twice with ethanol to remove any unreacted monomer and poly-L-lactide of low molecular weight, immersed in acetone for 24 hours to remove a trace amount of the remaining catalyst, and then dried. After the prepared poly-L-lactide/tricalcium phosphate composite powder was dried under vacuum at 60° C., it was introduced into a mold and compression-molded at about 190° C. for one hour. Thereafter, this was rapidly cooled to 100° C. and left to stand for several hours at room temperature. The compression-molded composite which had been formed into a cylinder-shaped billet of a 13 mm diameter was extruded a solid state at a drawing velocity of 10 mm/minute, thereby preparing a poly-L-lactide/tricalcium phosphate composite material.

### EXAMPLES 2-11

The procedure of Example 1 was repeated, except that the kind and content of the monomer, the inorganic fine particle, the catalyst and the dispersant, were the same as described in Table 1 below. However, in Example 7, the polymerization reaction was carried out according to the bulk polymerization method, after which the powdering, washing and drying of the resulting composite were carried out in the same manner as in Example 1.

TABLE 1

| | Monomer | | Inorganic fine particle | | | |
|---|---|---|---|---|---|---|
| Example | Kind | Amount (g) | Kind | Amount (g) | Catalyst | Dispersant |
| 2 | L- | 120 | TCP of less | 6 | Stannous | Silicon oil |
| | lactide | | than 100 nm | | octoate | |
| 3 | L- | 120 | TCP of less | 6 | Stannous | Silicon oil |
| | lactide | | than 500 nm | | octoate | |
| 4 | L- | 120 | TCP of less | 12 | Stannous | Silicon oil |
| | lactide | | than 100 nm | | octoate | |
| 5 | L- | 120 | TCP of less | 30 | Stannous | Silicon |
| | lactide | | than 100 nm | | octoate | oil |
| 6 | L- | 120 | TCP of less | 60 | Stannous | Silicon oil |
| | lactide | | than 100 nm | | octoate | |
| 7 | L- | 120 | TCP of less | 60 | Stannous | — |
| | lactide | | than 50 nm | | octoate | |
| 8 | L- | 120 | S-HA of 200 nm to | 6 | Stannous | Silicon oil |
| | lactide | | 2 μm | | octoate | |
| 9 | L- | 120 | CMP of 200 nm to | 6 | Stannous | Silicon oil |
| | lactide | | 2 μm | | octoate | |
| 10 | L- | 120 | CMP of 200 nm to | 12 | Stannous | Silicon oil |
| | lactide | | 2 μm | | octoate | |
| 11 | L- | 120 | CaAl of 1 μm | 12 | Stannous | Silicon oil |
| | lactide | | | | octoate | |

The molecular weight of the prepared polymer was determined using the following Mark-Houwink equation that shows the relation between intrinsic viscosity and molecular weight for linear poly-L-lactide (PLLA), and a change in molecular weight was calculated from viscosity-average molecular weight:

[η]=4.41×10⁻⁴·Mw^{0.72}(dl/g)

The intrinsic viscosity [η] was calculated by extrapolation from the viscosity of solutions at concentrations of 0.1 g/dl, 0.25 dl/g and 0.5 dl/g.

In order to measure the flexural strength of the prepared poly-L-lactide/tricalcium phosphate composite material, the rod-shaped composite which had been drawn was cut into 100 mm lengths and evaluated in three-point bending tests (ASTM D790-98). Also, the flexural strength of the rod-shaped sample according to the applied load was calculated according to the following equation:

Flexural strength=8FL/(π)d², where F is the applied load, L is the distance between support points, and d is the diameter of the sample.

The change in molecular weight and the flexural strength of the prepared poly-L-lactide/tricalcium phosphate after the forming step are indicated in Table 2 below.

TABLE 2

| | Viscosity (dl/g) | | | | | | |
|---|---|---|---|---|---|---|---|
| | | After | | Diameter | | | |
| | Immediately | solid- | Change in | after solid-state | Drawing | Flexural | Flexural |
| | after | state- | molecular | extrusion | ratio | strength | modulus |
| Example | polymerization | extrusion | weight | (mm) | (times) | (MPa) | (GPa) |
| 1 | 5.70 | 5.09 | 14.65%↓ | 4.524 | 6.98 | 352 | 12.7 |
| 2 | 5.45 | 4.98 | 11.73%↓ | 4.492 | 7.14 | 312 | 12.8 |
| 3 | 5.62 | 4.99 | 15.11%↓ | 4.461 | 7.24 | 357 | 13.0 |
| 4 | 5.85 | 5.20 | 15.00%↓ | 4.521 | 7.04 | 368 | 13.2 |
| 5 | 5.81 | 5.15 | 15.36%↓ | 4.505 | 7.09 | 340 | 13.6 |
| 6 | 5.10 | 4.63 | 12.65%↓ | 4.508 | 7.09 | 309 | 12.9 |
| 7 | 6.55 | 5.98 | 11.94%↓ | 4.477 | 7.18 | 302 | 12.8 |
| 8 | 4.47 | 4.01 | 13.97%↓ | 4.517 | 7.06 | 335 | 12.5 |
| 9 | 5.24 | 4.75 | 12.76%↓ | 4.459 | 7.24 | 284 | 12.9 |
| 10 | 5.07 | 4.49 | 15.40%↓ | 4.457 | 7.25 | 338 | 13.0 |
| 11 | 4.99 | 4.49 | 13.77%↓ | 4.495 | 7.13 | 313 | 12.9 |

### Comparative Example 1

Polymerization reaction was carried out using 120 g of L-lactide, dioctyltin and 300 ml of silicon oil of a 10 cSt viscosity under the same condition as in Example 1. As a result, a poly-L-lactide powder containing no inorganic fine particle was obtained. The obtained inorganic particle-free, poly-L-lactide powder was washed and dried, after which it was subjected to compression molding and solid-state extrusion in the same manner as in Example 1.

### Comparative Example 2

120 g of L-lactide and 0.27ml of a 20-fold dilution of stannous octoate were depressurized to completely remove toluene, and then heated to 200° C. so as to polymerize the L-lactide. After about 4 hours, when the load of a stirrer reached the climax, 50 g of anhydrous hydroxyapatite powder of less than 100 nm was introduced and then additionally stirred. The stirred material was cooled to terminate the polymerization reaction. In the same manner as in Example 7, the prepared poly-L-lactide/hydroxyapatite composite material was powdered, washed, compression-molded, and solid-state extruded.

### Comparative Example 3

In the same manner as in Comparative Example 2, L-lactide was melt polymerized. At the final step of the polymerization, 50 g of tricalcium phosphate of less than 100 nm was introduced and additionally stirred for 5 minutes, after which the polymerization was terminated. Then, in the same manner as in Example 7, the resulting material was powdered, washed, dried, compression-molded and solid-state extruded.

### Comparative Example 4

50 g of the inorganic particle-free, poly-L-lactide powder which had been obtained in the same manner as in Comparative Example 1, and 5 g of a tricalcium phosphate of less than 100 nm, were introduced into a Brabender twin-shaft kneader, and kneaded at 210° C. for 5 minutes under nitrogen atmosphere. Thereafter, in the same manner as in Example 7, the material was powdered, washed, and subjected to compression molding and solid-state extrusion.

### Comparative Example 5

50 g of the inorganic particle-free, poly-L-lactide powder which had been obtained in the same manner as in Comparative Example 1, and 5 g of a tricalcium phosphate of less than 100 nm, were sufficiently mixed with each other. The resulting material was subjected to compression molding and solid-state extrusion in the same manner as in Example 1.

### Comparative Example 6

10 g of the inorganic particle-free, poly-L-lactide powder which had been obtained in the same manner as in Comparative Example 1 was dissolved in 200 ml of chloroform, and a suspension which had been prepared by dispersing 1 g of a tricalcium phosphate particle of less than 100 mu in 100 ml of chloroform was slowly added thereto with stirring. Then, the tricalcium phosphate was sufficiently dispersed using an ultrasonic vessel. The poly-L-lactide/tricalcium phosphate suspension was added dropwise to excess methanol. After the precipitate was filtered, washed and dried, it was subjected to compression molding and solid-state extrusion in the same manner as in Example 1.

### Comparative Example 7

50 g of L-lactide and 40 g of tricalcium phosphate of less than 50 nm were mixed with each other. In the same manner as in Example 7, the mixture was polymerized, powdered, washed, dried, compression-molded and solid-state extruded.

The change in molecular weight and the flexural strength of the prepared poly-L-lactide/tricalcium phosphate composite material after the forming step are indicated in Table 3 below.

TABLE 3

| | | | | Diameter | | | |
|---|---|---|---|---|---|---|---|
| | Viscosity (dl/g) | | | after | | | |
| | Before | After | Change | solid- | | | |
| | mixing | mixing | in | state | Drawing | Flexural | Flexural |
| Comparative | of | of | molecular | extrusion | ratio | strength | modulus |
| Example | particle | particle | weight | (mm) | (times) | (MPa) | (GPa) |
| 1 | 5.14 | — | — | 4.452 | 7.26 | 245 | 11.7 |
| 2 | 2.75 | 2.04 | 33.81%↓ | 4.532 | 7.01 | 64.2 | 5.62 |
| 3 | 2.64 | 1.90 | 36.74%↓ | 4.519 | 7.05 | 71.0 | 6.23 |
| 4 | 6.20 | 3.87 | 47.97%↓ | 4.503 | 7.10 | 168 | 10.8 |
| 5 | 5.47 | [symbol] | — | 4.490 | 7.14 | 196 | 12.0 |
| 6 | 5.62 | 4.58 | 24.77%↓ | 4.516 | 7.06 | 188 | 11.5 |
| 7 | 5.33 | [symbol] | — | 9.487 | 1.60 | 205 | 17.5 |

As apparent from Comparative Examples as described above, the material containing only the prior biodegradable polymer exhibited an insufficient flexural strength of 245 (MPa) (Comparative Example 1). Thus, in order to solve this insufficient strength, there are known methods for preparing the composite material consisting of the polymer and the biodegradable inorganic fine particle. These methods include a method where the polylactide as the polymer is melt-blended with the fine particle (Comparative Example 2); a method where the inorganic fine particle is introduced at the final step of melt polymerization (Comparative Example 3); a method where the mixing is carried out in a powder state (Comparative Example 5); and a method where the mixing is carried out in a solution state (Comparative Example 6). However, the prior methods according to Comparative Examples mostly exhibited the rapid reduction of about 24 to 48% in molecular weight as compared to the initial molecular weight. In addition, they exhibited a mechanical strength of less than 200 Mpa, indicating that there is no improvement in the mechanical strength.

On the contrary, in Examples of the present invention, the polymerization reaction was carried out after the inorganic fine particle was added to and uniformly dispersed in the organic monomer. For this reason, the dispersibility of particle, which is exceptionally excellent as compared to the case where the polymer is mixed with the particle, could be obtained. Also, in Example of the present invention, the polymer did not undergo the molecular chain cleavage phenomenon, and thus, it was possible to prepare the composite material having excellent strength.

Moreover, from Comparative Example 7, it could be found that, where an excess amount of the inorganic fine particle is used, the strength of the composite was reduced as compared to the case where fine particle is not used. Also, it could be found that, at the fine particle content of 0.5 to 60% by weight, a reduction in molecular weight of the polymer was lowered and the strength-reinforcing effect was increased.

### Industrial Applicability

As apparent from the foregoing, the present invention provides the method for easily preparing the high-strength composite material, which comprises polymerizing the organic monomer after mixing and dispersing the inorganic fine particle in the organic monomer. According to the method of the present invention, the dispersibility of the fine particle is greatly improved, so that the reinforcing effect of the particle is remarkably increased. Thus, the high-strength material for bone fixation which has the mechanical strength of the desired level can be prepared.

Furthermore, in the composite material according to the present invention, the biocompatible inorganic particle is used as the reinforcing material, so that the rapid recovery of injured bone tissues can be promoted and also the long-term side effect can be reduced.

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A method for manufacturing a high-strength, biodegradable, organic polymer/inorganic particle composite material for bone fixation, which comprises the steps of:
mixing and dispersing a biocompatible inorganic fine particle having an average particle size of less than 2 μm, at the amount of 0.5 to 60% by weight in a biodegradable organic monomer;
polymerizing said biodegradable organic monomer, thereby preparing a biodegradable organic polymer/inorganic particle composite; and
forming said biodegradable organic polymer/inorganic particle composite into a desired shape.

2. The method of claim 1, in which said biodegradable organic monomer is selected from the group consisting of glycolide, DL-lactide, L-lactide, caprolactone, dioxanone, esteramide, oxalate and mixtures thereof.

3. (deleted)

4. The method of claim 1, in which said inorganic fine particle is a calcium phosphorus compound or a calcium aluminate compound.

5. The method of claim 4, in which said calcium phosphorus compound is one selected from the group consisting of hydroxyapatite (HA), tricalcium phosphate (TCP), and calcium metaphosphate (CMP).

6. (deleted)

7. The method of claim 1, in which said polymerization step is carried out using a method selected from dispersion polymerization, suspension polymerization, bulk polymerization or melt polymerization.

8. The method of claim 7, in which said dispersion polymerization is carried out using an alkyl/aryl-substituted siloxane compound or a long-chain saturated hydrocarbon compound, as a dispersant.

9. The method of claim 7, in which said suspension polymerization is carried out using an alkyl/aryl-substituted siloxane compound or a long-chain saturated hydrocarbon compound, as a dispersant.

10. The method of claim 1, in which said forming step is carried out using at least one selected from the group consisting of injection molding, compression molding and solid-state extrusion.

11. The method of claim 10, in which said compression molding is carried out at a temperature of 150 to 240° C., if the biodegradable organic polymer is poly-L-lactide.

12. (deleted)

13. The method of claim 10, in which said solid-state extrusion is conducted at a temperature of 130 to 150° C. and a drawing ratio of 5 to 12 times, if said biodegradable organic polymer is poly-L-lactide.

14. A high-strength, biodegradable, organic polymer/inorganic particle composite material for bone fixation having a flexural strength of more than 250 MPa, which is prepared according to the method of claim 1.
